(19) United States
(12) Patent Application Publication
(10) Pub. No.: US 2022/0192483 A1
(43) Pub. Date: Jun. 23, 2022

(54) COMPUTERIZED SELF GUIDED VISION MEASUREMENT

(71) Applicant: 0869316 B.C. Ltd. dba Vision Pros, Surrey (CA)

(72) Inventor: Steven LEE, Chicago, IL (US)

(21) Appl. No.: 17/553,777

(22) Filed: Dec. 16, 2021

Related U.S. Application Data
(60) Provisional application No. 63/127,056, filed on Dec. 17, 2020.

(51) Int. Cl. A61B 3/032; A61B 3/00

(52) U.S. Cl. CPC A61B 3/032; A61B 3/0041; A61B 3/0033

(57) ABSTRACT

Self-guided computerized vision testing system wherein the user is able to complete the test in a local or remote location, generating non clinical data that is delivered to a separate professional corporation or physician for potential medical interpretation, judgement, or analysis.

### TECHNICAL FIELD

Methods and systems are provided for self-guided computerized vision measurement, where a user can assess their own visual function. Particular embodiments provided computer-based systems and methods for users to quantify their own vision attributes.

### BACKGROUND

Eyesight is responsible for over 80% of learning in an individual. Visual performance of an individual is useful and important to measure to determine and differentiate changes in the individual's visual function. In the traditional method of testing the visual function of an individual, an optometrist, ophthalmologist, or an optician would typically use sophisticated and correspondingly expensive optical tools or devices to assess and analyze, inter alia, the health of the front and back of the eye, the ability for the eye to detect different colors, the ability for the eye(s) to move and focus together, and measurements of how well an individual can see both objectively and subjectively.

Many conditions or disease processes of the human eye can manifest as conditions such as diabetic retinopathy, glaucoma, macular degeneration, amblyopia, cataracts, retinoblastoma, myopia, hyperopia, astigmatism, presbyopia, diplopia, ptosis, dry eye syndrome, and/or the like. While some such conditions can be treated and monitored effectively using current tools such as slit lamps, binocular indirect ophthalmoscopes, phoropters, and visual field machines, there is a general desire, with respect to vision function, to create a measurement stasis and to determine differentials from that level of stasis. There is a general desire for assessment of vision that can be performable by users without the need for expensive optical assessment equipment. Such assessment may permit personalized tracking of changes. The traditional mechanisms or tools that are diagnostic or analytical (e.g. a specific diagnostic measurement of the refractive power of an eye at +1.00) are costly. In consequence, under the traditional model, significant time gaps could exist from one diagnosis to the next, with no supplementary information about the visual function in between the diagnoses. On the other hand, if a person's visual function is stable, periodic complete diagnoses may be unnecessary. It may suffice to only check if there has been any change in the visual function from one measurement to the next. Therefore, there is a general desire for a cost-effective self-guided assessment to allow users to determine if their own visual function is changing on a more frequent basis. In the age of information and data analytics alongside numerous human performance tracking applications and tools, such assessment tool may be valuable.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides computerized self-guided methods for vision assessment which allow individuals without access to eye-care services to undergo a self-guided vision testing solution by using their computer and mobile device. The solution replicates many of the positive experiences of a clinical practice, wherein a user will experience questions probed in an audio format, as well as the ability to slowly answer questions pertaining to their vision in a self-guided speed, so as to ensure they feel comfortable with their responses. The vision testing solution analyzes four components of a typical vision exam: acuity, sphere, cylinder, and axis; but does so in a manner of raw data accumulation, with potential clinical determinations done by interpretation and analysis by a licensed physician.

Another aspect of the invention provides a method for assessing vision of a user. The method comprises: guiding the user to a suitable distance from a display of a computer using one or more outputs from a mobile device, the mobile device operatively connected to the computer; and, separately, for each eye of the user: determining a magnitude of a spherical measurement for the user, wherein determining the magnitude of the spherical measurement for the user comprises testing for an acuity optotype by: presenting, by the computer, at least one diagram directly to the eye of the user via the display; and enabling the user to select, via interaction with the mobile device, at least one input per diagram, wherein the at least one input per diagram corresponds to an acuity measurement.

Using the one or more outputs from the mobile device may comprise using audio prompts.

Guiding the user to a specified distance from the display may comprise causing the mobile device to execute an application, which uses a camera of the mobile device to assist the user in determining whether the user is at the suitable distance from the display. The application may comprise a web-based application that does not require the user to download a separate application.

Assisting the user in determining whether the user is at the suitable distance from the display may comprise: displaying a shape on the display; capturing an image of the displayed shape using the camera of the mobile device; and guiding the user to move away from or toward the display until the captured image of the displayed shape is of a size which corresponds to the suitable distance from the display.

Guiding the user to move away from or toward the display may comprise: displaying a window on the mobile device; guiding the user toward the display if the captured image of the displayed shape is smaller than the window; and guiding the user away from the display if the captured image of the displayed shape is larger than the window.

The method may comprise enforcing the user to maintain the suitable distance from the display during the step of determining the magnitude of the spherical measurement for the user. Enforcing the user to maintain the suitable distance from the display may comprise: performing the step of guiding the user to the suitable distance from the display a plurality of times during the step of determining the magnitude of the spherical measurement for the user; and if, for any instance of the performance of the step of guiding the user to the suitable distance from the display, the user is not located at the suitable distance, then discontinuing the step of determining the magnitude of the spherical measurement for the user until the user is located at the suitable distance.

The method may comprise providing the user with instructions for performing the vision test via output from a mobile device that is operatively connected to the computer.

The method may comprise determining whether the user is correctly following the instructions using a camera of the mobile device.

Determining whether the user is correcting following the instructions using the camera of the mobile device may comprise capturing an image of the user using the camera of the mobile device and determining whether the user is covering an appropriate one of the user's eyes based on the image of the user.

Determining whether the user is covering an appropriate one of the user's eyes based on the image of the user may comprise using an artificial intelligence-based classifier.

The method may comprise detecting when the user is at a suitable distance from the display for determining the magnitude of the spherical measurement for the user.

The method may comprise detecting when the user has moved away from the suitable distance from the display.

The method may comprise: determining vision assessment raw results, the vision assessment raw results being in a non-conventional format for eye-care practitioners; and transmitting the vision assessment raw results to an optical professional or organization of optical professionals for determining if analysis or conversion can be made from the vision assessment raw results to a clinically usable format. The optical professional or organization of optical professionals may be different from an operator of the computer or a provider of the method.

Another aspect of the invention provides a method for determining when a user is correctly following instructions in a computer conducted vision test, whereby the user is able to respond to the computer via a tactile input to a mobile device being held in their hand or via an audio input to the computer or to the mobile device using their voice.

Another aspect of the invention provides a method for assessing vision of a user. The method comprises: separately, for each eye of the user: presenting, by a computer, at least one diagram directly to the eye of the user via a display of the computer; and enabling the user to select at least one input per diagram using a handheld mobile device from a location spaced apart from the display, the handheld mobile device operatively connected to the computer. The method also comprises ensuring that the user is complying with instructions for the vision assessment using feedback output from the handheld mobile device.

Ensuring that the user is complying with the instructions may comprise ensuring that the user is spaced apart from the display by a suitable distance using information obtained from a camera of the mobile device. Ensuring that the user is complying with the instructions may comprise ensuring that the user has properly covered one of their eyes using information obtained from a camera of the mobile device.

Ensuring that the user is spaced apart from the display by the suitable distance may comprise: displaying a shape on the display; capturing an image of the displayed shape using the camera of the mobile device; and guiding the user to move away from or toward the display until the captured image of the displayed shape is of a size which corresponds to the suitable distance from the display.

Another aspect of the invention provides a method for determining how far a user is from their screen when a user is utilizing a remotely conducted vision test via the internet.

Another aspect of the invention provides a method for determining and detecting when a user is not in their correct desired position from their computer screen when undergoing a remotely conducted vision test.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a flow chart of a method for providing computer-implemented self-guided testing of user visual function according to a particular embodiment of this invention.

FIG. 2 depicts a schematic view of a user conducting vision test by positioning their body at a distance away from a computer screen and controlling a mobile phone as a remote control for the operation of the vision testing system and method.

FIG. 3 illustrates a flow chart of an example acuity test displaying a Landolt C ring according to an embodiment of this invention.

FIG. 4 illustrates a flow chart of an example axis test displaying a line test and/or a fan wheel according to an embodiment of this invention.

FIG. 5 illustrates a flow chart of an example cylinder test displaying circles of various line thickness according to an embodiment of this invention.

FIG. 6 illustrates a flow chart of an example stage of gathering medical history and sending the gathered medical history to a professional for review according to an embodiment of this invention.

FIG. 7 depicts two example data sets each containing results from a respective vision test performed by a user to be reviewed by an optical professional.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

Traditional optometry systems for assessing eye refraction are typically embodied as individual tabletop systems. In traditional tabletop optometry systems, an auto-refractor can be used to determine objectively the refraction of the eye. In addition to such objective eye-refraction measurement, one can also carry out so-called subjective refraction determinations in which different lenses or other optical elements with different optical properties are given to a subject (for example, in an eyeglass frame with a succulent mount) to permit the subject to express their preference for particular sets of optical elements over others. During subjective refraction testing, a subject typically sees an eye chart containing characters or symbols placed at a relatively large distance, 20 feet from their eye or from their body for instance. As a consequence, a relatively large amount of space is conventionally needed for such subjective refractive assessment. In addition, the equipment required for both objective and subjective refractive testing is often relatively costly. There is a general desire for assessment of vision that can be performable by users without the need for expensive optical assessment equipment.

Aspects of the present innovation provide methods and systems for self-guided computerized vision measurement, where a user can assess their own visual function. Particular embodiments provide computer-based systems and methods for users to quantify their own vision attributes. In some such embodiments, recommendations may be provided for refractive correction of the eye. In some particular embodiments, recommendations may be provided for the correction of myopia, hyperopia and/or presbyopia.

Refractive assessment of the human eye may be characterized in two general categories. The first category is a traditional method of vision assessment wherein errors in the eyes and/or cylinders are assessed using subjective refraction measurements. The second category is vision assessment using wavefront analysis that is capable of assessing a variety of aberrations, including some or all of focus error, cylinder error, spherical aberration, coma, and others, using an objective wave-front sensor. For either of these assessment categories, conventional vision correction approaches are conceptually limited to the correction of single focus and cylinder defects. In addition, the procedure of measuring the eye is often constrained and complicated by subjective factors related to the determination of refractive errors in the eye, and in particular when attempting to measure the ocular cylinder error. Cylindrical error is also known as astigmatism, which can be made up of both near- and far-sighted spherical and astigmatic power.

There are at least five limiting factors for traditional subjective refraction measurements using traditional optometry equipment. Firstly, subjective refraction measurements are restricted to the discrete number of lenses available in the phoropter, since subjective refraction measurements use subjective evaluation of such lenses to assess vision. Focus error is typically limited to 0.125 diopter (D) resolution (increment) and cylinder error is typically limited to 0.25D resolution. Second, in a refraction correction greater than −2.00 D, a small variance of the cylinder axis (e.g. within a few degrees) may cause substantial differences in efficacy because it is more difficult to subjectively see a difference when there is only a small amount of cylinder power in refractive error. Therefore, the subjective determination of the cylinder axis can be troublesome and challenging to pinpoint. Third, since subjective refractive measurement requires the patient's subjective response to multiple refractive corrections and the practitioner's examination of those subjective reactions, human error can be problematic. Fourth, subjective refraction measurements are inherently empirical and therefore time consuming. Practitioners conducting subjective refraction measurements must evaluate the endpoint of the empirical refractive correction (e.g. by testing different combinations of lenses in person to determine the corrective refraction a patient needs to correct the patient's vision). This process is time consuming because subjective refraction measurement relies on human visual optimization control, with as many as three independent adjustments, including focus error, refractive power and cylinder axis. Moreover, the determination of the exact endpoint correction is known to differ among different practitioners even if they analyze the same patient.

Another downside associated with the use of traditional subjective refraction measurement composites is that existing lens manufacturing techniques (e.g. for correction of vision problems) require high tolerances and cause false vision correction spread. Error in standard vision correction procedure using subjective refraction calculation in the same eye prescription by various practitioners and rough resolution of approximately 0.25D refractive power are commonly prescribed for standard vision correction. This resolution limitation may trigger situations where major differences may occur between practitioners. As a result of these issues, ophthalmic lenses currently available in the ophthalmic industry are also typically limited to 0.25D resolution. Correction of eye astigmatism using conventional vision correction is further complicated by high resistance to accommodating unique prescriptions or fine distinctions between prescriptions among standard eyeglass lens manufacturing, especially when patients are given a prescription with the smallest unit of a cylindrical value, but lens manufacturers believe the prescription is not clinically perceivable. Given the limitation of lens manufacturing techniques, a self-guided assessment may offer as much value to a patient as a full diagnosis does.

Aspects of the invention provide methods and systems for computer-guided user self-assessment of subjective refraction.

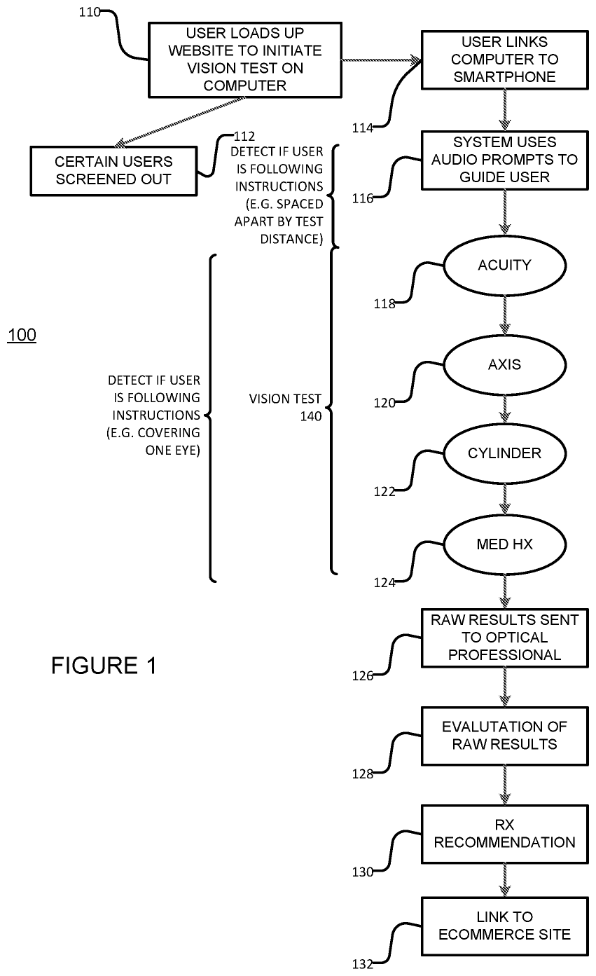

FIG. 1 is a schematic depiction of a method 100 for providing computer-implemented self-guided testing of user visual function according to a particular embodiment. Method 100 may be performed using a computer device and a mobile device (e.g. smart phone), which may be configured (e.g. by suitable programming) to work together to perform method 100. Other suitable computer-based devices may also be used to implement method 100. For example, virtual reality (VR), augmented reality (AR) and mixed reality (MR) devices such as VR, AR, MR headsets and controllers, etc. may also be used to implement method 100.

Method 100 begins in block 110, where a user accesses a website to initiate a vision test via their computer. Block 112 shows that certain users may be screened out from the vision test, for example, if they have certain computer parameters that are not compatible or otherwise suitable for performing the method 100 vision test (e.g. screen sizes that are too small and/or the like; or the user is unable to position herself for a suitable distance away from the computer screen). A user may also be screened out of the process if the devices (e.g. computer device and mobile device) are unable to connect to one another or if the user's mobile device camera is unable to be utilized. Block 114 shows that a user may connect their computer device to their mobile device, which may be done, for example, via a Quick Response (QR) code scan or any other suitable technique, such as a mobile phone application. Block 116 shows that method 100 of the illustrated embodiment may use audio prompts from the computer and/or the smartphone and may provide audio prompts to the user to instruct them how to conduct the vision test. Such audio prompting, while not necessary, may be useful for some users who may have a hard time seeing visual prompts when they are not wearing corrective eyewear.

A vision test 140 is then performed (as explained in more detail below). In some embodiments, a user may create a user account and store all of her past vision test results as well as other suitable information (e.g. Med HX 124, corrective eyewear purchase history, etc.) to the user account. Such information may be accessed to aid vision test 140. For example, a user's vision test 140 may be customized based on such information. Vision test 140 may include components for assessment of acuity test 118, astigmatism test 120, cylinder test 122 and Med HX (medical history) 124. During vision test 140, method 100 may check if the user is following instructions properly. These steps in vision test 140 will be discussed in more detail below.

Block 126 shows how the raw results of vision test 140 (which may be different from industry specific or an eye care industry type values) are generated and may be sent to a suitable optical professional (or organization), such as a doctor or a optometrist, for example. The optical professional(s) to whom the raw data are sent in block 126 may be affiliated with and/or non-affiliated with the provider of method 100. Block 128 shows that the block 126 raw data may be analyzed or otherwise evaluated by the optical professional(s). For example, such optical professional(s) may convert the block 126 raw results to typical industry values, if necessary or possible. Block 130 shows that a prescription may be provided (e.g. by the optical professionals used in blocks 126, 128) or by method 100 itself. Finally, in block 132, the user may be linked to an e-commerce site, where they can shop for corrective eyewear.

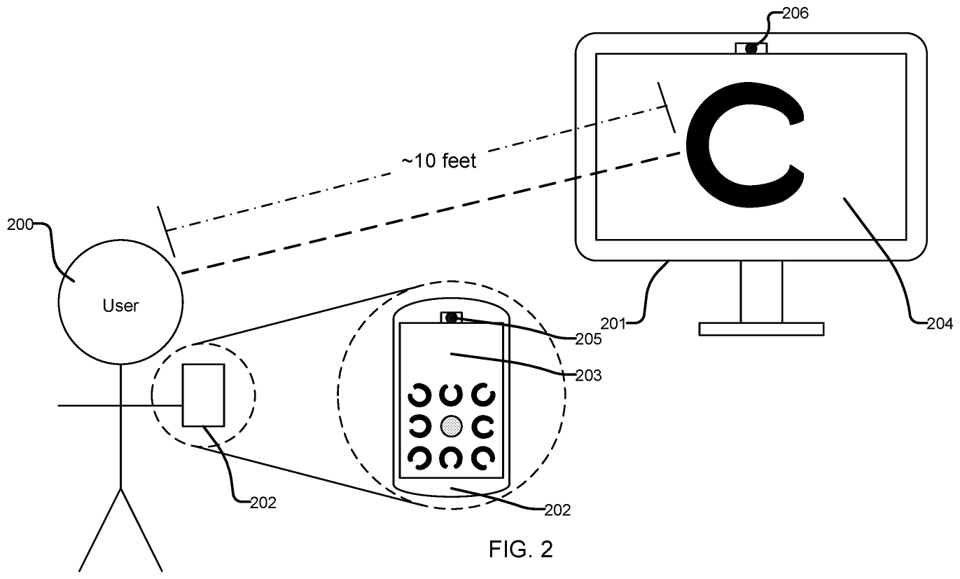

Vision test 140 and its implementation is now described in more detail. FIG. 2 depicts a schematic view of a user 200 conducting vision test 140. A user 200 controls a mobile device 202 which comprises a mobile screen 203. User 200 is positioned at a distance from a computer device 201 which comprises a computer screen or other suitable display 204 (referred to hereinafter without loss of generality as computer display 204). In some embodiments, user 200 is positioned about ten feet away from computer screen 204. In some embodiments, mobile device 202 is a mobile phone. Computer device 201 may comprise any suitable computer device(s). For example, a desktop computer, a laptop computer, a tablet computer and/or the like. In some embodiments, mobile device 202 comprises a front-facing camera 205. Computer device 201 may also comprise a front-facing camera 206.

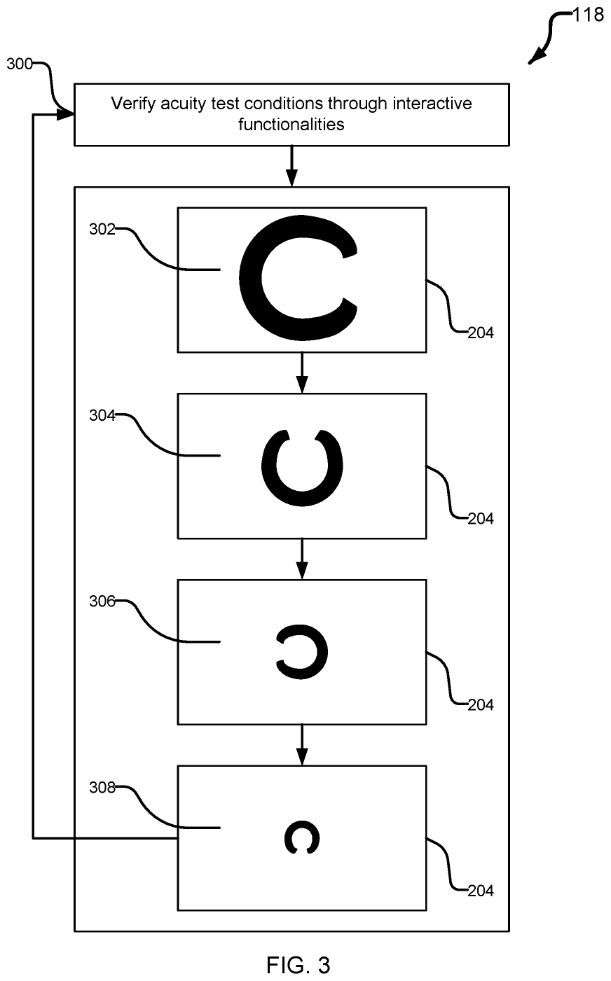

FIG. 3 illustrates a flow chart of an example acuity test 118 according to an embodiment of this invention. Acuity test 118 may be performed via a web based platform that connects mobile device 202 and computer device 201. The two devices (computer device 201 and mobile device 202) may be connected by allowing user 200 to first scan a QR code that is displayed on computer screen 204 using a camera (e.g. a rear-facing camera) of their mobile device 202 (e.g. in block 114 discussed above). When such a QR scan is performed (or the computer and mobile device 202 are otherwise connected), mobile device 202 may automatically display and/or prompt the user to open a website and/or application on their mobile device 202. Once this website and/or application is open on mobile device 202, method 100 connects computer device 201 to mobile device 202 (block 114). User 200 is then able to follow instructions of vision test 140 that are verbally spoken via their speakers and/or audio interfaces of computer device 201 and/or speakers and/or audio interfaces of mobile device 202. User 200 can click the buttons on mobile device 202 to answer the questions they are asked.

In acuity test 118, a subjective measurement of visual acuity is made to determine the baseline and clinical presentation of how well the user is seeing. Acuity test 118 may involve using subjective focus quantification using an interactive and random presentation display of a precisely calibrated Landolt C (or any other suitable indicia). FIG. 2 shows example visual displays on computer screen 204 and mobile screen 203 for one example step in an example acuity test 118. Computer screen 204 may display a Landolt ring of a chosen size and a chosen orientation. On mobile screen 203, a user interface displays a corresponding response control panel, allowing user 200 to select a response, which would be recorded and the records kept by method 100 (e.g. in a suitable memory device operably connected to computer device 201 and/or to mobile device 202). In some embodiments, the visual display may comprise a plurality of Landolt rings with different orientations. In one embodiment, the visual display comprises nine Landolt rings for user 200 to select the response from.

Returning to FIG. 3, acuity test 118 may begin with step 300 of verifying the acuity test conditions through interactive functionalities. Method 100 may not proceed to acuity test 118 unless the acuity test conditions are met. Computer device 201 and/or mobile device 202 may be utilized to implement the interactive functionalities of step 300. In one example embodiment of step 300, method 100 may verify that user 200 is at a suitable distance away from computer screen 204 or otherwise help to guide user 200 to the suitable distance from the computer screen 204. For example, computer device 201 may display a shape on computer screen 204 and mobile device 202 may display a digital window of the same shape on mobile screen 203. Computer device 201 and/or mobile device 202 may then instruct user 200 to fit the shape on computer screen 204 into the digital window on mobile screen 203 through audio prompts. User 200 would only be able to properly fit the shape into the digital window at the suitable distance from computer screen 204, thus achieving the desired distance if user 200 completes the task. In some embodiments, computer device 201 and/or mobile device 202 may access one or more operational parameters (e.g. magnification, zoom and/or the like) of the camera of mobile device 202 so as to choose the correct size of the shape to display on computer screen 204 and/or the screen of mobile device 202.

In another example embodiment of step 300, method 100 may verify that user 200 is conducting vision test 140 in a monocular fashion (i.e. covering one of the eyes). For example, the front-facing camera of computer device 201 may be activated to scan an image of user 200. Any suitable computer programs may be utilized to analyze the image to verify that user 200 is covering an eye correctly according to the instructions. The computer program may be an artificial intelligence computer vision program (e.g. facial recognition programs). In other embodiments, the computer vision program may scan an image of user 200 continuously to ensure user 200 properly covers the eye throughout the desired part of acuity test 118. The computer vision program may pause acuity test 118 if user 200 ceases to properly cover the eye and only resume after user 200 properly covers the eye according to method 100's instructions.

Acuity test 118 may then continue to a sequence of images for acuity measurement. FIG. 3 shows an example embodiment using the Landolt C indicia. However, any other suitable acuity indicia may be used. In the illustrated example, Landolt C ring(s) of various sizes and various orientations may be displayed on computer screen 204. In one embodiment, a single Landolt C ring may be displayed once at a time. In another embodiment, multiple Landolt C rings may be displayed.

In the illustrated example, acuity test 118 begins with step 302, which displays a large-sized Landolt C ring. User 200 looks at computer screen 204 and inputs a corresponding response onto mobile device 202 by means as discussed above. After user 200 inputs the response on mobile device 202, computer device 201 is notified of user 200's response and causes acuity test 118 to move onto the next image (e.g. image 304). Acuity test 118 may continue with successively smaller-sized Landolt C rings in each subsequent image (i.e. images 304, 306, 308 as shown in the illustrated example FIG. 3). However, this is not necessary. Acuity test 118 may present the Landolt C rings of any size and of any orientation in any sequence. It is also to be understood that acuity test 118 is not limited to any number of images in the sequence.

In some embodiments, when user 200 completes acuity test 118 for one eye, method 100 may cause acuity test 118 to loop back to step 300 and instruct user 200 to conduct acuity test 118 for the other eye. In other embodiments, method 100 may instruct user 200 to complete all tests (i.e. acuity 118, astigmatism 120, cylinder 122 and Med HX 124) for one eye before instructing user 200 to conduct the tests for the other eye.

Still in other embodiments, multiple indicia may be displayed simultaneously and/or sequentially and acuity test 118 may ask user 200 which image on computer screen 204 is sharper. The response (which may be input via mobile device 202) may involve swiping left/right to make a selection, touching the mobile device screen on the left/right to make a selection, pressing buttons on the left/right of the mobile device body to make a selection, or selecting an image on the computer display and then long pressing a button on the mobile device to make a selection, for example.

Once user 200 completes acuity test 118, user 200 may be prompted to do an axis test 120 to determine if they have any astigmatism by examining whether the user has an astigmatic axis. In astigmatism, axis, which is measured in degrees, refers to where on the cornea the astigmatism is located. Cylinder represents the amount of lens power one needs to correct for astigmatism. Therefore, axis test 120 is a screening test for any potential astigmatism.

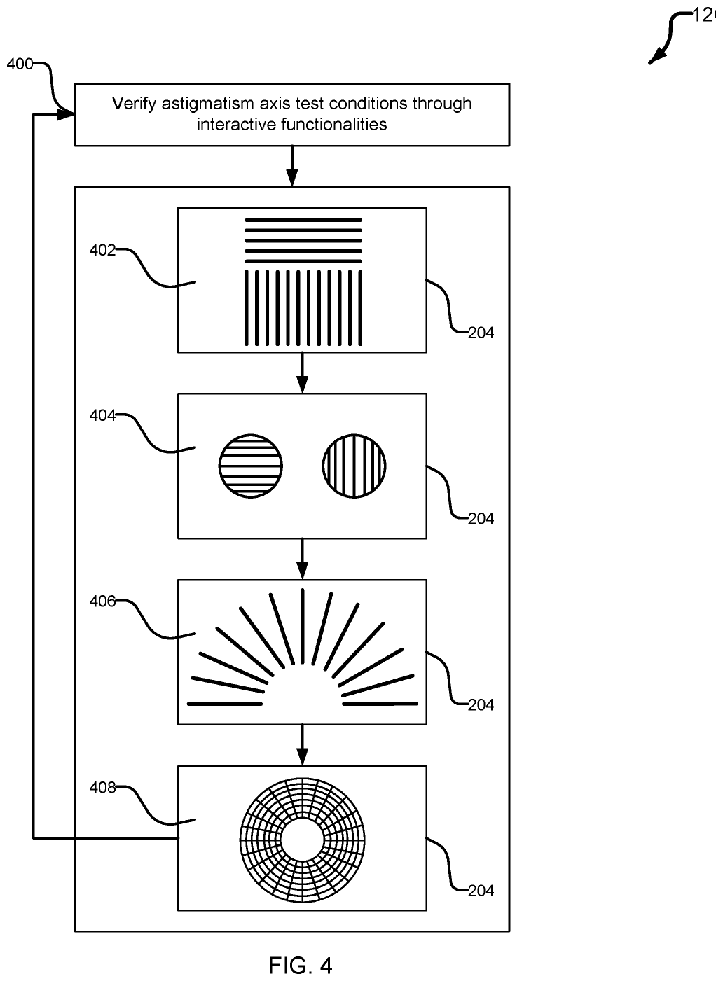

FIG. 4 illustrates a flow chart of an example axis test 120 according to an embodiment of this invention. Axis test begins with step 400 to verify that the conditions for axis test 120 are fully met. Step 400 may be carried out in similar fashion as step 300 of acuity test 118. For example, step 400 may instruct user 200 to maintain the desired distance and/or cover one of the eyes. After verifying the conditions at step 400, axis test 120 continues to by causing computer 201 and its display 204 to display a sequence of images for axis test of astigmatism.

Axis test 120 may utilize any suitable images for testing astigmatism axis. In the illustrated example, axis test 120 begins with a line test 402. Computer device 201 and/or mobile device 202 instruct user 200 to carefully observe the lines in line test 402. Then, user 200 may be prompted to provide a response to a question. In some embodiments, the question is posed audibly through the speakers of computer device 201 and/or mobile device 202. User 200 inputs a response to the question through interacting with mobile device 202. For example, the question may require a binary response (e.g. True or False; Left or Right) where response options are displayed on mobile screen 203. For example, in the illustrated example of the line test 402, the question may be whether the horizontal lines appear to have the same colour as the vertical lines or not. User 200 may select the answer by interacting with mobile device 202 in any suitable manners as discussed above.

Step 404 shows another example line test 404, the horizontal lines and vertical lines are each incorporated into a shape (e.g. a circle). The user may be asked to select which circle has lines that look sharper by an audio prompt. The corresponding response options may be presented on mobile screen 203. For example, the two circles in line test 404 may be replicated on mobile device screen 203. User 200 may then choose which circle looks sharper. In some embodiments, user 200 chooses the circle by tapping mobile screen 203 or swiping across mobile screen 203.

Other images for testing axis may include use of an astigmatism fan wheel. In the illustrated example, steps 406 and 408 show example variations of an astigmatism fan wheel. For steps 406 and 408, a question that may be posed to user 200 is whether all the lines in the images appear to have the same colour, or that some appear black and others appear grey. The question may require a binary response. User 200 may select the response according to methods discussed above. Similar to acuity test 118, after axis test 120 is complete for one eye, method 100 may loop back to step 400 of axis test 120 and instruct user 200 to complete axis test 120 for the other eye.

Steps 402, 404, 406 and 408 are presented in a particular sequence in FIG. 4. However, this specific sequence shown in FIG. 4 is not necessary. Steps 402, 404, 406 and 408 may be presented in any suitable sequences. It is also to be understood that axis test 120 is not limited to any number of images in the sequence. Axis test 120 allows method 100 to determine if the user has any astigmatic axis, and, if they do, axis test 120 testing also allows method 100 to determine the direction of astigmatism that the user has.

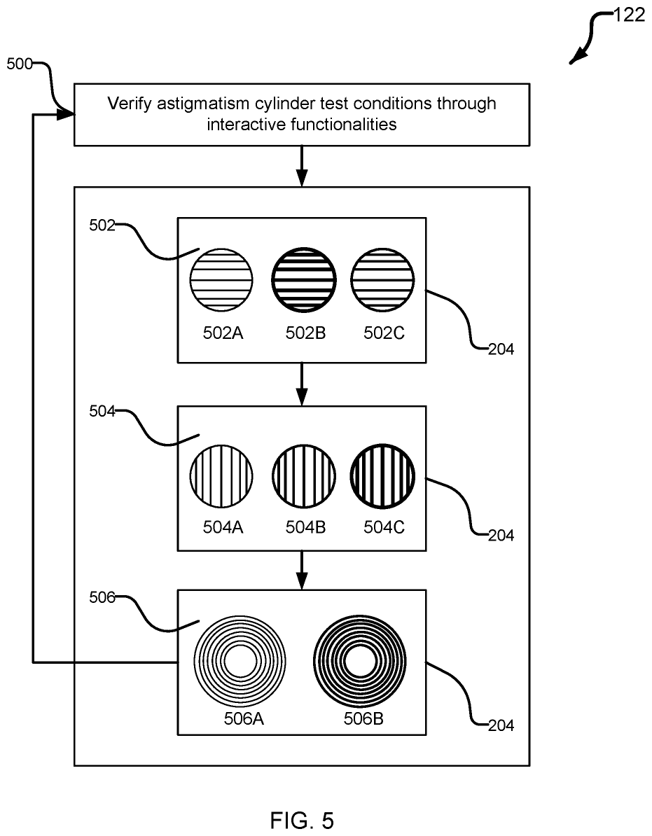

If axis test 120 finds an astigmatic axis for user 200, method 100 then proceeds to cylinder test 122 where the user is prompted to undergo a cylindrical measurement. If no astigmatic axis is found, method 100 may bypass cylinder test 122 and proceed directly to Med HX 124. FIG. 5 illustrates a flow chart of an example cylinder test 122 according to an embodiment of this invention. Similar to step 300 of acuity test 118 and step 400 of axis test 120, step 500 of cylinder test 122 verifies the conditions for a cylinder test through interactive functionalities as discussed above.

Similarly, cylinder test 122 involves display 204 of computer 201 presenting a sequence of images for cylinder measurement. In example step 502 of cylinder test 122, user 200 may be presented with three geometrically identical circles but with different line thickness on computer screen 204. Cylinder test 122 may ask user 200 to choose which of the circles looks like it has the most defined lines of the three. User 200 may respond according to the methods discussed above. The methods for answering cylinder test 122 may be suitably modified to permit user to select among three options (instead of two) by way of their mobile device 202. Additionally or alternatively, step 502 (or any other steps of cylinder test 122) may be modified to present inquiries which lead to binary answers.

In example step 504, circles with vertical lines are presented. The order in which the circles with different line thickness may be shuffled to be different from the order shown in example step 502. User 200 responds in similar fashions as in example step 502. In example step 506, another example cylinder test image is shown to have two circles with a plurality of concentric rings, each with a different line thickness. Similarly, user 200 may be prompted to select which circle appears more defined. Steps 502, 504 and 506 can be presented in any order. It is also to be understood cylinder test 122 may include any number of steps in its sequence. Cylinder test 122 allows for a numerical assessment of cylinder to be performed in a manner that allows the measurement to be recorded. As discussed in more detail below, this recorded data is not a direct dioptric link but may be analyzed by an optical professional to deduce the dioptric link.

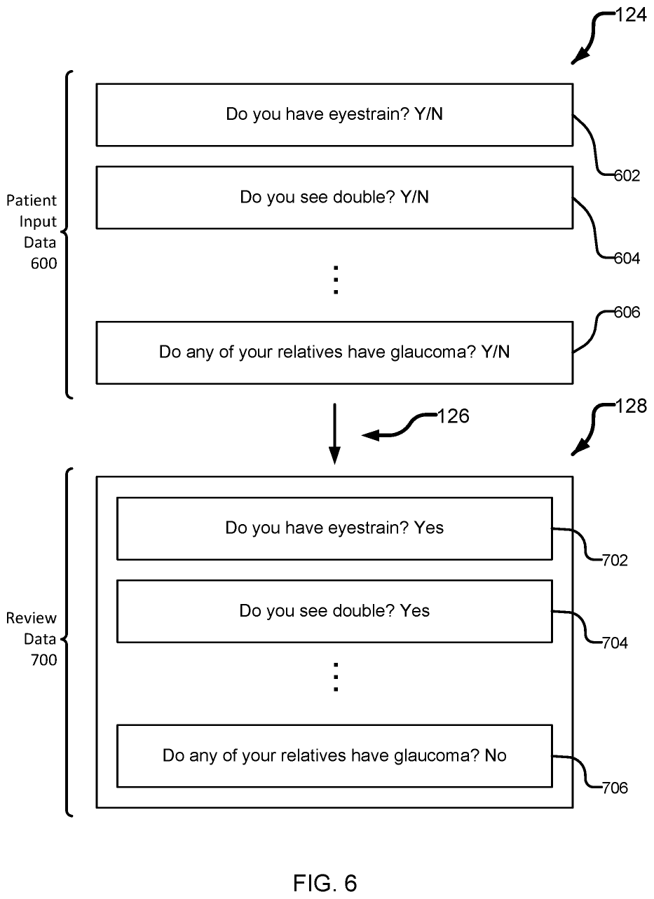

Med HX 124 of vision test 140 is a stage in which method 100 gathers medical history information from user 200. For Med HX 124, user 200 may answer a number of questions about their medical history, which may be incorporated into the raw data to be sent to the optical professional (block 126) and then evaluated by the optical professional. FIG. 6 illustrates a flow chart of an example sequence from Med HX 124 to block 128. Med HX 124 may comprises a number of questions presented sequentially to user 200. The questions may have controlled responses (e.g. binary answers like YES/NO or True/False), although this is not necessary. For example, Med HX 124 may inquire as to whether user 200 has eyestrain (block 602), whether user 200 sees double (block 604), whether user 200 (or his/her close relatives) have had glaucoma, diabetes, hypertension and/or the like (block 606), etc. User 200's responses are saved and stored along with the questions as raw patient input data 600.

After user 200 completes Med HX 124, the raw patient input data 600 may be sent to an optometry practitioner along with the results (not shown in FIG. 6) from acuity test 118, axis test 120 and cylinder test 122 (if any) in block 126. At block 128, an optometry practitioner may obtain and analyse review data set 700. Review data 700 includes user 200's response to questions in Med HX 124. For example, data entries 702, 704 and 706 each corresponds respectively to questions 602, 604 and 606. Review data 700 also includes the results (not shown in FIG. 6) from acuity test 118, axis test 120 and cylinder test 122 (if any).

After the completion of vision test 140, the user will have undergone an acuity test 118, an astigmatic axis test 120, a potential cylinder test 122, and a medical history review 124. While acuity test 118's measurement results in a direct Snellen equivalent (i.e. the diagnostic standards for acuity in current optometry practices) based on the distance the user is from computer screen 204, method 100 may use some assistance to determine cylinder test 122 and axis test 120 measurements. For cylinder test 122 and axis test 120 measurements, the raw results detected by method 100 may be sent (via any suitable interface) to an optical professional (e.g. a licensed optometrist or ophthalmologist) or a professional organization in block 126. The optical professional or professional organization may interpret the raw measurements (e.g. 35 mm for the size of the letter the user could see clearly at 10 feet away) and may translate that raw measurement into a proper diagnosis (e.g. a 20/30 vision diagnosis).

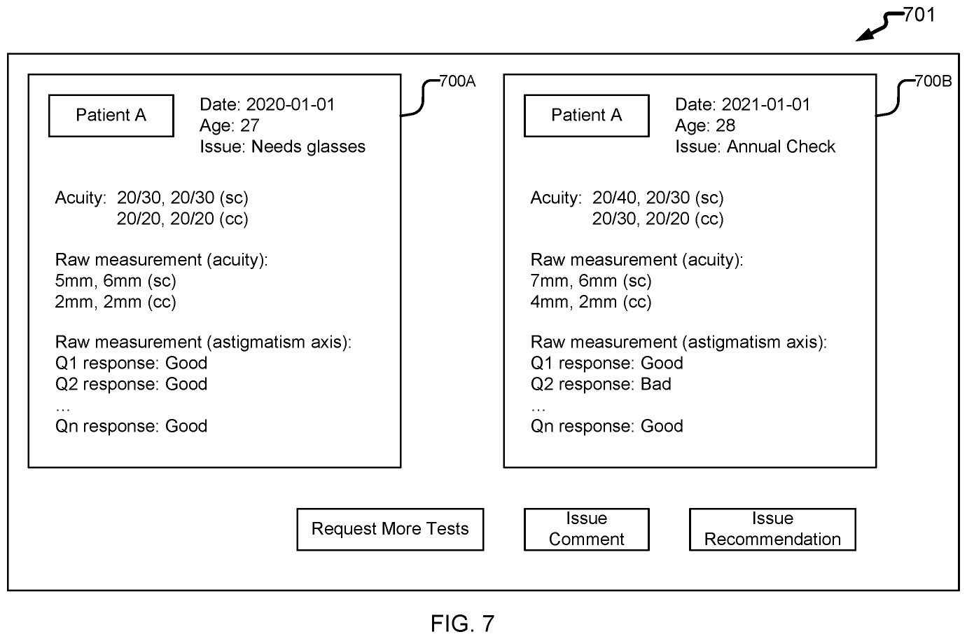

FIG. 7 depicts two example data sets each containing results from a respective vision test 140 performed by a user 200 to be reviewed by an optometry professional. FIG. 7 depicts an example display 701 of data sets on a working device (e.g. computer) of an optometry professional. FIG. 7 illustrates two data sets 700A and 700B created at separate times. Data sets 700A and 700B may be separated by any time period. In some embodiments, three or more datasets may be presented for evaluation of the eyes' change over time. The availability of data sets 700A and 700B allows the optical professional to evaluate the raw results of vision test 140 of a user 200 according to step 128 in method 100 (shown in FIG. 1).

In the illustrated example, data sets 700A and 700B show Patient A's acuity diagnosis (e.g. 20/20) and raw measurements (e.g. 5 mm), as well as the raw measurement of the astigmatism axis (e.g. records of Patient A's responses to questions). The acuity diagnosis may be automatically calculated from the raw measurements of acuity. The optical professional analyzes the results and translates raw measurements into diagnosis if necessary. Furthermore, records of the raw measurements from data sets 700A and 700B also allow the optical professional to determine the change in Patient A's eyes. For this purpose, the earlier data set 700A may be considered as comprising the baseline measurements for comparison to the later measurement resulting in data set 700B.

In the illustrated example, Patient A's acuity measurement becomes worse in one of the eyes. For example, from 20/30 to 20/40 corresponding to a raw measurement of 5 mm to 7 mm. In addition, the astigmatism axis measurements also have some differences between data sets 700A and 700B. The optical professional may request more tests for Patient A. The request may be communicated to Patient A by any suitable means. In other embodiments with other possible data sets, the optical professional may deduce that a patient has developed astigmatism. The optical professional may also issue comments for Patient A regarding Patient A's eye health and discuss the implications from the measurements. Furthermore, the optical professional may also issue a recommendation to Patient A about the kind of corrective method Patient A could undertake. In some embodiments, the recommendation may be related to corrective eyewear.

### Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”)). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control circuit for a device may implement methods as described herein by executing software instructions in a program memory accessible to the processors.

Processing may be centralized or distributed. Where processing is distributed, information including software and/or data may be kept centrally or distributed. Such information may be exchanged between different functional units by way of a communications network, such as a Local Area Network (LAN), Wide Area Network (WAN), or the Internet, wired or wireless data links, electromagnetic signals, or other data communication channel.

For example, while processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed at different times.

In addition, while elements are at times shown as being performed sequentially, they may instead be performed simultaneously or in different sequences. It is therefore intended that the following claims are interpreted to include all such variations as are within their intended scope.

Software and other modules may reside on servers, workstations, personal computers, tablet computers, image data encoders, image data decoders, PDAs, color-grading tools, video projectors, audio-visual receivers, displays (such as televisions), digital cinema projectors, media players, and other devices suitable for the purposes described herein. Those skilled in the relevant art will appreciate that aspects of the system can be practised with other communications, data processing, or computer system configurations, including: Internet appliances, hand-held devices (including personal digital assistants (PDAs)), wearable computers, all manner of cellular or mobile phones, multi-processor systems, microprocessor-based or programmable consumer electronics (e.g., video projectors, audio-visual receivers, displays, such as televisions, and the like), set-top boxes, color-grading tools, network PCs, mini-computers, mainframe computers, and the like.

The invention may also be provided in the form of a program product. The program product may comprise any non-transitory medium which carries a set of computer-readable instructions which, when executed by a data processor, cause the data processor to execute a method of the invention. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

In some embodiments, the invention may be implemented in software. For greater clarity, “software” includes any instructions executed on a processor, and may include (but is not limited to) firmware, resident software, microcode, and the like. Both processing hardware and software may be centralized or distributed (or a combination thereof), in whole or in part, as known to those skilled in the art. For example, software and other modules may be accessible via local memory, via a network, via a browser or other application in a distributed computing context, or via other means suitable for the purposes described above.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

Various features are described herein as being present in “some embodiments”. Such features are not mandatory and may not be present in all embodiments. Embodiments of the invention may include zero, any one or any combination of two or more of such features. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible).

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

1. A method for assessing vision of a user, the method comprising:
guiding the user to a suitable distance from a display of a computer using one or more outputs from a mobile device, the mobile device operatively connected to the computer; and,
separately, for each eye of the user, determining a magnitude of a spherical measurement for the user, wherein determining the magnitude of the spherical measurement for the user comprises testing for an acuity optotype by:
presenting, by the computer, at least one diagram directly to the eye of the user via the display; and
enabling the user to select, via interaction with the mobile device, at least one input per diagram, wherein the at least one input per diagram corresponds to an acuity measurement.

2. The method of claim 1 wherein using the one or more outputs from the mobile device comprises using audio prompts.

3. The method of claim 1 wherein guiding the user to a specified distance from the display comprises causing the mobile device to execute an application, which uses a camera of the mobile device to assist the user in determining whether the user is at the suitable distance from the display.

4. The method of claim 3 wherein the application is a web-based application that does not require the user to download a separate application.

5. The method of claim 3 wherein assisting the user in determining whether the user is at the suitable distance from the display comprises:
displaying a shape on the display;
capturing an image of the displayed shape using the camera of the mobile device; and
guiding the user to move away from or toward the display until the captured image of the displayed shape is of a size which corresponds to the suitable distance from the display.

6. The method of claim 5 wherein guiding the user to move away from or toward the display comprises:
displaying a window on the mobile device;
guiding the user toward the display if the captured image of the displayed shape is smaller than the window; and
guiding the user away from the display if the captured image of the displayed shape is larger than the window.

7. The method of claim 3 wherein the method comprises enforcing the user to maintain the suitable distance from the display during the step of determining the magnitude of the spherical measurement for the user, wherein enforcing the user to maintain the suitable distance from the display comprises:
performing the step of guiding the user to the suitable distance from the display a plurality of times during the step of determining the magnitude of the spherical measurement for the user; and
if, for any instance of the performance of the step of guiding the user to the suitable distance from the display, the user is not located at the suitable distance, then discontinuing the step of determining the magnitude of the spherical measurement for the user until the user is located at the suitable distance.

8. The method of claim 1 comprising providing the user with instructions for performing the vision test via output from a mobile device, the mobile device operatively connected to the computer.

9. The method of claim 8 comprising determining whether the user is correctly following the instructions using a camera of the mobile device.

10. The method of claim 8, wherein determining whether the user is correcting following the instructions using the camera of the mobile device comprises capturing an image of the user using the camera of the mobile device and determining whether the user is covering an appropriate one of the user's eyes based on the image of the user.

11. The method of claim 10 wherein determining whether the user is covering an appropriate one of the user's eyes based on the image of the user comprises using an artificial intelligence-based classifier.

12. The method of claim 1 comprising detecting when the user is at a suitable distance from the display for determining the magnitude of the spherical measurement for the user.

13. The method of claim 12 comprising detecting when the user has moved away from the suitable distance from the display.

14. The method of claim 1 comprising:
determining vision assessment raw results, the vision assessment raw results being in a non-conventional format for eye-care practitioners; and
transmitting the vision assessment raw results to an optical professional or organization of optical professionals for determining if analysis or conversion can be made from the vision assessment raw results to a clinically usable format.

15. The method of claim 14 wherein the optical professional or organization of optical professionals is different from an operator of the computer or a provider of the method.

16. A method for determining when a user is correctly following instructions in a computer conducted vision test, whereby the user is able to respond to the computer via a tactile input to a mobile device being held in their hand or via an audio input to the computer or to the mobile device using their voice.

17. A method for assessing vision of a user, the method comprising:
separately, for each eye of the user:
presenting, by a computer, at least one diagram directly to the eye of the user via a display of the computer; and
enabling the user to select at least one input per diagram using a handheld mobile device from a location spaced apart from the display, the handheld mobile device operatively connected to the computer; and
ensuring that the user is complying with instructions for the vision assessment using feedback output from the handheld mobile device.

18. The method of claim 17 wherein ensuring that the user is complying with the instructions comprises ensuring that the user is spaced apart from the display by a suitable distance using information obtained from a camera of the mobile device.

19. The method of claim 17 wherein ensuring that the user is complying with the instructions comprises ensuring that the user has properly covered one of their eyes using information obtained from a camera of the mobile device.

20. The method of claim 18 wherein ensuring that the user is spaced apart from the display by the suitable distance comprises:
displaying a shape on the display;
capturing an image of the displayed shape using the camera of the mobile device; and
guiding the user to move away from or toward the display until the captured image of the displayed shape is of a size which corresponds to the suitable distance from the display.
